(19) United States
(12) Patent Application Publication
(10) Pub. No.: US 2005/0147573 A1
(43) Pub. Date: Jul. 7, 2005

(54) Deodorant compositions

(75) Inventors: Kyoko Abe, Chib-ken (JP); Naoto Uyama, Shiroi City (JP); Hiromichi Sakaguchi, Chiba City (JP)

Correspondence Address: NOVOZYMES NORTH AMERICA, INC., 500 FIFTH AVENUE, SUITE 1600, NEW YORK, NY, 10110, US

(73) Assignee: NOVOZYMES A/S, BAGSVAERD (DK)

(21) Appl. No.: 10/512,223

(22) PCT Filed: Apr. 24, 2003
(86) PCT No.: PCT/DK03/00270

(51) Int. Cl. A61K 7/32; A61K 7/06

(52) U.S. Cl. 424/65; 424/74

(57) ABSTRACT

The present invention relates to methods and compositions for reducing odor from a locus by contacting the locus with an enzymatic agent, an oxidizing agent and a shredded, sliced or crushed plant material which naturally contains one or more phenolic compounds.

### FIELD OF THE INVENTION

The present invention relates to enzymatic methods and compositions capable of reducing odors from human, animal and industrial sources.

### BACKGROUND

Various environmental odors are part of our daily life, such as refrigerator odors and odors from pets and domestic animals. Because some of these odors are unpleasant to humans, a wide variety of approaches have been proposed to eliminate these odors. One such conventional deodorizing method is to adsorb the substance causing the offensive odor. Activated carbon is an example of such an adsorbent. However, as activated carbon is not easy to handle and dispose of in large amounts, a naturally based environmentally friendly alternative is desirable. The present invention provides such an alternative.

### SUMMARY

The present invention provides a deodorant composition comprising an enzymatic agent, an oxidizing agent, and shredded, sliced or crushed plant material which naturally contains one or more phenolic compounds.

In a second aspect the invention provides a method of reducing odor from a locus, comprising contacting the locus with the composition of the invention.

### Definitions

In the context of the present invention the term “odor” is intended to mean a bad smell as perceived by an average human being.

The term “odorant” means a substance yielding a bad smell (odor). An odorant precursor is a substance which may be transformed into an odorant by e.g. biochemical reactions.

The term “deodorant” means a substance, which is capable of reducing odor.

### DETAILED DESCRIPTION

Enzymatic Agent

The enzymatic agent of the invention comprises a phenol oxidizing enzyme and an oxidizing agent.

In a preferred embodiment the enzymatic agent further comprises one or more other enzymatic activities, such as proteases, lipases, cutinases, amylases, carbohydrases, cellulases, pectinases, mannanases, arabinases, galactanases and/or xylanases.

Phenol Oxidizing Enzymes

In the context of the present invention the phenol oxidizing enzyme is an enzyme or a compound exhibiting phenol oxidizing activity. The phenol oxidizing enzyme may be a peroxidase or a compound exhibiting peroxidase activity, a laccase or a compound exhibiting laccase activity, a catechol oxidase. (EC 1.10.3.1), an o-aminophenol oxidase (EC 1.10.3.4), or a bilirubin oxidase (EC 1.3.3.5).

The phenol oxidizing enzyme of the invention may typically be present in concentrations of from 1 to 10000 microgram enzyme protein per liter aqueous solution, preferably of from 5 to 2000 microgram enzyme protein per liter aqueous solution, more preferably of from 5 to 1000 microgram enzyme protein per liter aqueous solution, and most preferably of from 1 to 500 microgram enzyme protein per liter aqueous solution.

Assays for determining the activity of these enzymes are well known to persons of ordinary skill in the art.

It is to be understood that phenol oxidizing enzyme variants (e.g. produced by recombinant techniques) are included within the meaning of the term “phenol oxidizing enzyme”.

Laccases and Compounds Exhibiting Laccase Activity

Compounds exhibiting laccase activity may be any laccase enzyme comprised by the enzyme classification EC 1.10.3.2, or any fragment derived therefrom, exhibiting laccase activity.

Preferred laccase enzymes and/or compounds exhibiting laccase activity are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinus, e.g., C. cinereus, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radita (WO 92/01046), or Coriolus, e.g., C. hirsutus (JP 2-238885).

Suitable examples from bacteria include a laccase derivable from a strain of Bacillus.

A laccase derived from Coprinus, Myceliophthora, Polyporus, Scytalidium or Rhizoctonia is preferred; in particular a laccase derived from Coprinus cinereus, Myceliophthora thermophila, Polyporus pinsitus, Scytalidium thermophilum or Rhizoctonia solani.

The laccase or the laccase related enzyme may furthermore be one which is producible by a method comprising cultivating a host cell transformed with a recombinant DNA vector which carries a DNA sequence encoding said laccase as well as DNA sequences encoding functions permitting the expression of the DNA sequence encoding the laccase, in a culture medium under conditions permitting the expression of the laccase enzyme, and recovering the laccase from the culture.

Determination of Laccase Activity (LACU)

Laccase activity (particularly suitable for Polyporus laccases) may be determined from the oxidation of syringaldazin under aerobic conditions. The violet colour produced is photometered at 530 nm. The analytical conditions are 19 mM syringaldazin, 23 mM acetate buffer, pH 5.5, 30° C., 1 min. reaction time.

1 laccase unit (LACU) is the amount of enzyme that catalyses the conversion of 1.0 mmole syringaldazin per minute at these conditions.

Determination of Laccase Activity (LAMU)

Laccase activity may be determined from the oxidation of syringaldazin under aerobic conditions. The violet colour produced is measured at 530 nm. The analytical conditions are 19 mM syringaldazin, 23 mM Tris/maleate buffer, pH 7.5, 30° C., 1 min. reaction time.

1 laccase unit (LAMU) is the amount of enzyme that catalyses the conversion of 1.0 mmole syringaldazin per minute at these conditions.

Peroxidases and Compounds Exhibiting Peroxidase Activity

Compounds exhibiting peroxidase activity may be any peroxidase enzyme comprised by the enzyme classification (EC 1.11.1.7), or any fragment derived therefrom, exhibiting peroxidase activity. In the context of this invention, compounds exhibiting peroxidase activity comprise peroxidase enzymes and peroxidase active fragments derived from cytochromes, haemoglobin or peroxidase enzymes.

Preferably, the peroxidase employed in the composition of the invention is producible by plants (e.g. horseradish or soybean peroxidase) or microorganisms such as fungi or bacteria.

Some preferred fungi include strains belonging to the subdivision Deuteromycotina, class Hyphomycetes, e.g., Fusarium, Humicola, Trichoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucaria (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g., Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g., T. versicolor (e.g. PR4 28-A).

Further preferred fungi include strains belonging to the subdivision Zygomycotina, class Mycoraceae, e.g., Rhizopus or Mucor, in particular Mucor hiemalis.

Some preferred bacteria include strains of the order Actinomycetales, e.g. Streptomyces spheroides (ATTC 23965), Streptomyces thermoviolaceus (IFO 12382) or Streptoverticillum verticillium ssp. verticillium.

Other preferred bacteria include Bacillus pumilus (ATCC 12905), Bacillus stearothermophilus, Rhodobacter sphaeroides, Rhodomonas palustri, Streptococcus lactis, Pseudomonas purrocinia (ATCC 15958) or Pseudomonas fluorescens (NRRL B-11).

Further preferred bacteria include strains belonging to Myxococcus, e.g., M. virescens.

The peroxidase may furthermore be one which is producible by a method comprising cultivating a host cell transformed with a recombinant DNA vector which carries a DNA sequence encoding said peroxidase as well as DNA sequences encoding functions permitting the expression of the DNA sequence encoding the peroxidase, in a culture medium under conditions permitting the expression of the peroxidase and recovering the peroxidase from the culture.

Particularly, a recombinantly produced peroxidase is a peroxidase derived from a Coprinus sp., in particular C. macrorhizus or C. cinereus according to WO 92/16634.

Determination of Peroxidase Activity (POXU)

One peroxidase unit (POXU) is the amount of enzyme which under the following conditions catalyze the conversion of 1 micromole hydrogen peroxide per minute: 0.1 M phosphate buffer pH 7.0, 0.88 mM hydrogen peroxide, 1.67 mM 2,2′-azino-bis(3-ethylbenzothiazoline-6-sulfonate) (ABTS) and 30° C.

The reaction is followed for 60 seconds (15 seconds after mixing) by the change in absorbance at 418 nm, which should be in the range 0.15 to 0.30.

For calculation of activity is used an absorption coefficient of oxidized ABTS of 36 mM⁻¹ cm⁻¹ and a stoichiometry of one micromole H₂O₂ converted per two micromole ABTS oxidized.

Oxidizing Agent

If the phenol oxidizing enzyme requires a source of hydrogen peroxide as an oxidizing agent to exhibit phenol oxidizing activity (e.g. peroxidase activity), the source may be hydrogen peroxide or a hydrogen peroxide precursor for in situ production of hydrogen peroxide. Any solid entity which liberates upon dissolution a peroxide which is useable by the phenol oxidizing enzyme can serve as the source of hydrogen peroxide. Compounds which yield hydrogen peroxide upon dissolution in water or an appropriate aqueous based medium include but are not limited to metal peroxides, percarbonates, persulphates, perphosphates, peroxyacids, alkyperoxides, acylperoxides, peroxyesters, urea peroxide, perborates and peroxycarboxylic acids or salts thereof. Any compound which generates a peroxide that the phenol oxidizing enzyme can use to exhibit phenol oxidizing activity (e.g. peroxidase activity) is an acceptable source of hydrogen peroxide for this invention; this includes a large number of compounds as one skilled in the art will recognize. Mixtures of two or more of these substances can also be used.

Another source of hydrogen peroxide is a hydrogen peroxide generating enzyme system, such as an oxidase together with a substrate for the oxidase. Examples of combinations of oxidase and substrate comprise, but are not limited to, amino acid oxidase (see e.g. U.S. Pat. No. 6,248,575) and a suitable amino acid, glucose oxidase (see e.g. WO 95/29996) and glucose, lactate oxidase and lactate, galactose oxidase (see e.g. WO 00/50606) and galactose, and aldose oxidase (see e.g. WO 99/31990) and a suitable aldose.

By studying EC 1.1.3._, EC 1.2.3._, EC 1.4.3._, and EC 1.5.3._ or similar classes (under the International Union of Biochemistry), other examples of such combinations of oxidases and substrates are easily recognized by one skilled in the art.

The source of hydrogen peroxide may be added at the beginning of or during the process, e.g., typically in an amount corresponding to levels of from 0.001 mM to 25 mM, preferably to levels of from 0.005 mM to 5 mM, and particularly to levels of from 0.01 to 1 mM.

If the phenol oxidizing enzyme requires a source of molecular oxygen as an oxidizing agent to exhibit phenol oxidizing activity (e.g. laccase activity), the source of oxygen may be oxygen from the atmosphere or an oxygen precursor for in situ production of oxygen. Oxygen from the atmosphere will usually be present in sufficient quantity. If more O₂ is needed, additional oxygen may be added, e.g. as pressurized atmospheric air or as pure pressurized O₂.

Plant Material

In the context of the present invention the plant material is any shredded, sliced, cut or crushed plant items.

The plant material may be provided on the basis of frozen materials, heat treated materials (such as tea or coffee residues), fresh materials, fermented materials, or provided in any other form known in the art. The extract may contain the plant material, e.g. the plant material may be mashed, pulped or homogenized.

According to the present invention the plant material may be obtainable from e.g. fruits, vegetables, cereals, leaves, wood or any other plant items.

Useful fruits comprise, but are not limited to, pomes and seed fruits (apples, pears, etc.), grapes, tomatoes, citrus (orange, lemon, lime, mandarin), prunes, cherries, black currants, red currants, raspberries, strawberries, cranberries, pineapple, and any other tropical fruit.

Useful vegetables comprise, but are not limited to, potatoes, carrots, celery, and onions.

Useful cereals comprise, but are not limited to, wheat and maize.

Useful leaves comprise, but are not limited to, tea leaves (e.g. green tea leaves). The tea leaves may be residues from a tea brewing process.

The plant material may also comprise a mixture of plant items from the same plant, or more than one plant material from different plants.

The plant material may have been extracted with hot water, such as a tea leaf residue. Hot water means water with a temperature in the range of 60-120 degrees Celsius, preferably in the range of 70-110 degrees Celsius, more preferable in the range of 80-100 degrees Celsius. Water at a temperature above 100 degrees Celsius must be kept under pressure (above 1 atmosphere).

The plant material of the invention naturally contains one or more phenolic compounds. Examples of such natural phenolic compounds include, but are not limited to, lignin, lignosulfonic acid, humic acid, nitrohumic acid, tannin, catechin, gallic acid, urishiol, hesperidin, guaiachol, 4-methyl-guaiachol, 4-ethyl-guaiachol hinokitiol.

Compositions

The present invention provides a deodorant composition comprising:
an enzymatic agent comprising a phenol oxidizing enzyme and an oxidizing agent; and
shredded, sliced or crushed plant material which naturally contains one or more phenolic compounds.

The deodorant composition may be formulated in the form of a slurry, a suspension, a paste, a solid or a dry product formulation. The dry product formulation may subsequently be re-hydrated to form an active composition usable in the method of the invention.

When the composition is formulated as a dry formulation, the components may be mixed, arranged in discrete layers or packaged separately.

When formulated as a solid, all components may be mixed together, e.g. as a powder.

When other than dry form compositions are used and even in that case, it is preferred to use a two-part formulation system having the enzyme(s) separate from the rest of the composition.

The composition of the invention may further comprise auxiliary agents such as wetting agents, thickening agents, buffer, stabilisers, perfume, colourants, fillers and the like.

Useful wetting agents are surfactants, i.e. non-ionic, anionic, amphoteric or zwitterionic surfactants.

The composition of the invention may be a concentrated product or a ready-to-use product. In use, the concentrated product is typically diluted with water to provide a medium having an effective odor reducing activity, applied to the locus to be deodorized, and allowed to react with the odorants or odorant precursors present.

Methods and Uses

The present invention provides a method of reducing odor from a locus, comprising contacting the locus with:
an enzymatic agent comprising a phenol oxidizing enzyme and an oxidizing agent; and
shredded, sliced or crushed plant material which naturally contains one or more phenolic compounds.

The locus may be contacted with the composition of the invention by immersing the locus in an aqueous formulation of the composition (e.g. a suspension), by spraying the locus with the composition, by coating the locus with the composition using e.g. a cloth, a brush, a shovel, or the like. The composition may also be mixed with objects containing odorants. The locus is contacted with the composition in such a way that the odorants of the locus can react with the composition. Any method of applying the composition to a locus, which results in reducing the odor from the locus, is an acceptable method of application. The skilled person will easily recognize such methods.

In another embodiment the locus is contacted with the composition at between 5 and 80 degrees Celsius, preferably between 10 and 50 degrees Celsius, more preferably between 15 and 40 degrees Celsius, and most preferably at room temperature.

In another embodiment the locus is contacted with the composition at between pH 2 and pH 10, preferably at between pH 3.5 and pH 9, more preferably between pH 5 and pH 9.

In yet another embodiment the phenol oxidizing enzyme and the oxidizing agent are a laccase and a source of oxygen, or a peroxidase and a source of hydrogen peroxide.

The method of the invention may be capable of reducing the odor from a locus to less than 80% (preferably less than 50%, more preferably less than 30%, most preferably less than 20%, and in particular less than 10%) after 2 days at 20 degrees Celsius and 60-90% relative humidity, compared to an identical surface which has not been subjected to the method of the invention.

Evaluation of Odor

Odor may be evaluated by a trained sensory panel of at least 5 persons. The odor intensity is indicated on a scale from 0 to 10 where 0 equals ‘no odor’ and 10 equals ‘very strong odor’. All evaluations are performed twice, and average values are calculated.

Alternatively, as bad smelling compounds (odorants) are often volatile compounds, such as methyl mercaptan, traditional analysis methods, such as gas chromatography and gas detection tubes, may be used to detect and quantify these compounds.

The present invention is further described by the following examples which should not be construed as limiting the scope of the invention.

### EXAMPLES

Chemicals used as buffers and substrates were commercial products of at least reagent grade.

### Example 1

### Deodorant Composition with Laccase and Tea Leaf Residue

Enzyme: Myceliophthora thermophila laccase, activity: 276 LAMU/g (disclosed in patent application WO9533836 and available from Novozymes A/S, Denmark).

Tea: Green Tea (dry form), brand name “maccha iri ryokucha” (ITO EN, Ltd., Japan).

Odor Detector

Gas sampling pump kit: GV-100S (Gastec Corporation)

Gas detection tube: No. 71H, methyl mercaptan (Gastec Corporation)

Preparation of Green Tea Residue

400 ml water (80 degrees Celsius) was mixed with 20 g of green tea. After waiting for 40 seconds, the water was removed by using a cotton fiber filter.

Preparation of Standard Odor Solution

Methyl mercaptan, sodium salt, approx. 15% in water (Tokyo Kasei kogyo Co., Ltd.) was diluted with water in a ratio of 1:100.

Experimental Methods

In four 50 ml glass test tubes green tea residue was mixed with standard odor solution and enzyme in amounts as indicated in Tables 1-4, and mixed well. The test tubes were sealed with plastic film and incubated at 25 degrees Celsius for 1 hour. 50 microliter from each test tube was sampled and analyzed by using a Gas detection tube (No. 71H, attached to a Gas sampling pump: GV-100S) by reading the color graduation on the test tube (ppm of methyl mercaptan). The experiment was repeated four times.

Results

The “Deodorant efficiency” values in Tables 1-4 show clearly that the combination of a laccase and a plant material (green tea residue) is very efficient in removing the odorant (methyl mercaptan).

TABLE 1

| | Standard | Green tea | Laccase | Gas | Deodorant |
|---|---|---|---|---|---|
| Test tube | odor solution | residue | solution | detector | efficiency |
| no. | (microliter) | (g) | (microliter) | reading | (%) |
| 1 | 100 | 0 | 100 | 140 | 0 |
| 2 | 100 | 0.5 | 0 | 140 | 0 |
| 3 | 100 | 0.5 | 50 | 25 | 82 |
| 4 | 100 | 0.5 | 100 | 5 | 96 |

TABLE 2

| | Standard | Green tea | Laccase | Gas | Deodorant |
|---|---|---|---|---|---|
| Test tube | odor solution | residue | solution | detector | efficiency |
| no. | (microliter) | (g) | (microliter) | reading | (%) |
| 1 | 100 | 0 | 100 | 150 | 0 |
| 2 | 100 | 0.5 | 0 | 150 | 0 |
| 3 | 100 | 0.5 | 50 | 25 | 83 |
| 4 | 100 | 0.5 | 100 | 8 | 95 |

TABLE 3

| | Standard | Green tea | Laccase | Gas | Deodorant |
|---|---|---|---|---|---|
| Test tube | odor solution | residue | solution | detector | efficiency |
| no. | (microliter) | (g) | (microliter) | reading | (%) |
| 1 | 100 | 0 | 100 | 150 | 0 |
| 2 | 100 | 0.5 | 0 | 150 | 0 |
| 3 | 100 | 0.5 | 50 | 30 | 80 |
| 4 | 100 | 0.5 | 100 | 7 | 95 |

TABLE 4

| | Standard | Green tea | Laccase | Gas | Deodorant |
|---|---|---|---|---|---|
| Test tube | odor solution | residue | solution | detector | efficiency |
| no. | (microliter) | (g) | (microliter) | reading | (%) |
| 1 | 100 | 0 | 100 | 140 | 0 |
| 2 | 100 | 0.5 | 0 | 140 | 0 |
| 3 | 100 | 0.5 | 50 | 25 | 82 |
| 4 | 100 | 0.5 | 100 | 5 | 96 |

1. A deodorant composition comprising:
an enzymatic agent comprising a phenol oxidizing enzyme and an oxidizing agent; and
shredded, sliced or crushed plant material which naturally contains one or more phenolic compounds.

2. The composition of claim 1, wherein the plant material is wood, tea, coffee, or buckwheat.

3. The composition of claim 1, wherein the plant material has been extracted with hot water.

4. A method of reducing odor from a locus, comprising contacting the locus with the composition of claim 1.

5. (canceled)
